(19) United States
(12) Patent Application Publication
(10) Pub. No.: US 2026/0232878 A1
(43) Pub. Date: Aug. 13, 2026

(54) ABSORBABLE MARKER WIRE

(71) Applicant: Fort Wayne Metals Research Products, LLC, Fort Wayne, IN (US)

(72) Inventors: Adam J. Griebel, Hoagland, IN (US); Jeremy E. Schaffer, Fort Wayne, IN (US)

(21) Appl. No.: 19/049,280

(22) Filed: Feb. 10, 2025

(51) Int. Cl. A61L 31/18; A61L 31/02; A61L 31/08; A61L 31/10; A61L 31/14

(52) U.S. Cl. CPC A61L 31/18; A61L 31/022; A61L 31/088; A61L 31/10; A61L 31/148

(57) ABSTRACT

A medical device is formed primarily of wires with limited radiopacity, such as magnesium, and has at least one fully absorbable radiopaque marker wire to enhance the radiopacity of the device. The fully absorbable radiopaque marker wire may include one or more elements with greater radiopacity than the primary wire, including zinc, iron, molybdenum, or tungsten. To prevent galvanic interactions between the marker wires and the magnesium wires, an absorbable polymeric coating may be applied to the absorbable marker wire.

### BACKGROUND

### 1. Technical Field

The present disclosure relates to marker wires used to impart radiopacity to a medical device and relates in particular to absorbable marker wires for absorbable medical devices.

### 2. Description of the Related Art

Minimally invasive surgical procedures in which devices are delivered to the target site via guidewires and catheters often rely on real-time imaging methods such as fluoroscopy to enable accurate placement and ensure proper deployment. To be distinguished from fluoroscopic image background, devices must have a certain amount of radiopacity, or ability to block and scatter x-rays. Radiopacity is thus an important property and can be challenging to achieve in thin material sections, such as in fine wire products of the type used in medical devices including stents and blood filters, for example.

Radiopacity is largely a function of three attributes: section thickness, material density, and material atomic number. For small devices like stents with very thin struts (e.g. 0.3 to 0.02 mm), proper material selection is required to account for lack of section thickness. Based on the properties in Table 1 below, for example, material selection can have a large impact on device radiopacity. Magnesium is a promising absorbable metal for vascular applications, but the low density renders it essentially invisible under fluoroscopy. Other proposed absorbable metals (Zn, Fe, Mo) have higher densities and radiopacities. None of these metals have the radiopacity of common marker materials tantalum and platinum, but molybdenum is much more radiopaque than the other absorbable classes and is nearly 6 times the density of magnesium.

TABLE 1
Material properties of medically relevant metals

| | Density | Major Element |
|---|---|---|
| Alloy | (g/cc) | Atomic Number |
| Magnesium | 1.74 | 12 |
| Zinc | 7.14 | 30 |
| Iron | 7.87 | 26 |
| Molybdenum | 10.22 | 42 |
| Tantalum | 16.65 | 73 |
| Platinum | 21.45 | 78 |

Due to the excellent biological response associated with magnesium implants, it is desirable to employ medical implants comprised primarily of a magnesium-based alloy, though such implants are essentially invisible under fluoroscopy.

This problem has been addressed in laser-cut-tube magnesium-based stents on the market today through the addition of galvanically-isolated and radiopaque tantalum markers on the ends of the device (U.S. Pat. No. 8,992,600), such as those found on the Magmaris® or Freesolve® resorbable magnesium scaffolds produced by Biotronik. These markers remain in the patient indefinitely. Employing a similar strategy in a wire-based magnesium stent is challenging due to the geometrical restrictions associated with wire compared to laser-cut tube. Incorporation of permanent polymer-coated tantalum or platinum marker wires into a magnesium braid can provide device visibility (U.S. Patent Application Publication No. 20230157852), but leaving behind full wire segments can lead to later complications associated with wire migration.

What is needed is an improvement over the foregoing.

### SUMMARY

The present disclosure provides a medical device comprised primarily of wires with limited radiopacity, such as magnesium, and has at least one fully absorbable radiopaque marker wire to enhance the radiopacity of the device. The fully absorbable radiopaque marker wire may be comprised of one or more elements with greater radiopacity than the primary wire, including zinc, iron, molybdenum, or tungsten. To prevent galvanic interactions between the marker wires and the magnesium wires, an absorbable polymeric coating may be applied to the absorbable marker wire.

In one form thereof, the present disclosure provides an absorbable polymer coated radiopaque marker wire for radiopacity enhancement of a fully or partially absorbable medical device.

In another form thereof, the present disclosure provides a medical device formed primarily of absorbable marker wires to provide whole device visibility.

In a further form thereof, the present disclosure provides a method for producing a fully absorbable and partially radiopaque medical device with acceptable radiopacity and biological response, the method comprising braiding a combination of magnesium-based wires and fully absorbable polymer coated radiopaque marker wires, the wires fused together by heating the braid on the braiding mandrel at a sufficient temperature to fuse the polymer at the crossing points, providing a scaffold with enhanced radial stiffness and strength.

### BRIEF DESCRIPTION OF THE DRAWINGS

The above mentioned and other features of this invention, and the manner of attaining them, will become more apparent and the invention itself will be better understood by reference to the following description of embodiments of the invention taken in conjunction with the accompanying drawings, where:

FIG. 1 corresponds to Example 1 and is a schematic cross-section of a fully absorbable polymer coated radiopaque marker wire.

FIG. 2 corresponds to Example 1 and is a photograph of wire pairs of both magnesium paired with bare molybdenum and magnesium paired with polycaprolactone-coated molybdenum after immersion in a saline solution for 10 minutes.

FIG. 3 corresponds to Example 2 and is a photograph of a magnesium wire braid in which a PCL-coated molybdenum wire is threaded through. The image was acquired after 24 hour immersion in saline solution.

FIG. 4 corresponds to Example 3 and is a photograph a fully absorbable wire braid, the braid consisting of 24 wires, including 10 bare Mg wires, 10 PCL-coated Mg wires, and 4 PCL-coated Mo wires. The braid was heat treated to fuse the PCL crossing points.

FIG. 5 corresponds to Example 3 and is a photograph of the braid shown in FIG. 4 after immersion in a saline solution for 14 days, revealing no galvanic acceleration.

FIG. 6 corresponds to Example 3 and is an X-ray photograph of the braid shown in FIG. 4, demonstrating the visibility of the fully absorbable polymer coated marker wires compared to the magnesium wires (not visible).

Corresponding reference characters indicate corresponding parts throughout the several views. Unless stated otherwise the drawings are to scale and proportional.

### DETAILED DESCRIPTION

The embodiments disclosed below are not intended to be exhaustive or to limit the invention to the precise forms disclosed in the following detailed description. Rather, the embodiments are chosen and described so that others skilled in the art may utilize their teachings.

As used herein, “wire” or “wire product” encompasses continuous wire and wire products which may be continuously produced and wound onto a spool for later dispensation and use, such as wire having a round cross section and wire having a non-round cross section, including flat wire or ribbon. “Wire” or “wire product” also encompasses other wire-based products such as strands, cables, coil, and tubing, which may be produced at a particular length depending on a particular application. Although round cross-sectional wire forms are shown in the Figures of the present application and described further below, non-round wire forms may also be produced in accordance with the present disclosure. Exemplary non-round forms include polygonal cross-sectional shapes such as rectangular cross-sectional shapes.

“DFT®” is a registered trademark of Fort Wayne Metals Research Products LLC of Fort Wayne, IN, and refers to a bimetal or poly-metal composite wire product including two or more concentric layers of materials, typically at least one outer layer or shell disposed over a core filament and formed by drawing a tube or multiple tube layers over a core element.

“Absorbable”, “bioabsorbable”, “degradable”, “biodegradable”, “resorbable”, and “bioresorbable” are all terms used to indicate the ability of a device or material to structurally degrade and have its degradation products be excreted, metabolized, or assimilated by cells and/or tissues. See ASTM F3268 Appendix X1 for further discussion.

### 1. Fully Absorbable Radiopaque Marker Wire

Magnesium alloys are very promising for absorbable stents and other medical devices due to their high biocompatibility but are severely limited in terms of radiopacity (ability to be seen under x-ray fluoroscopy). To compensate, markers made of radiopaque material (e.g. Ta, Pt) are typically added to allow for proper delivery. These can be small disc-shaped markers in laser-cut tube stents or as entire wire elements in braided wire stents. In either case, the radiopaque marker material is much more noble than magnesium and so is coated with an inert polymer to avoid galvanic interactions. This marker material will remain in the body indefinitely, even after the rest of the device has been absorbed into the body. This has the potential to cause issues with later migration if the markers are not properly constrained.

It is desirable to have a radiopaque marker wire which would dissolve over time. Molybdenum (Mo) has recently been identified as a potential absorbable material which degrades slowly, and in our testing appears to be radiopaque enough to serve as a marker. See Griebel et. al., Bioactive Materials, Volume 40, 2024, pp 74-87, which is incorporated by reference herein in its entirety.

Other potential marker wire materials could include zinc (Zn), Zn alloys, iron (Fe), iron alloys, tungsten (W), Mo alloys, W alloys, and DFT composites with an Fe alloy shell and a Mo alloy core. To prevent galvanic corrosion, the marker wire is jacketed with an absorbable polymer like polycaprolactone (PCL), polydioxanone (PDO), poly (lactic-co-glycolic acid) (PLGA), polylactic acid (PLA), poly-1-lactic acid (PLLA), poly (glycerol sebacate) (PGS), or any number of known absorbable polymers.

Further, non-absorbable polymeric coatings like polyimide or PTFE which often contain natural porosity could be used. These polymers would be left behind indefinitely but would likely allow the Mo to degraded slowly over time. These polymers may also be more resilient to the braiding and heat treating processes and provide a better galvanic barrier than the absorbable polymers.

Due to the absorbable coating and the inherent degradation rates of the metal, the primary wires comprised of magnesium will degrade before the secondary marker wires. Without the constraint of the magnesium wires, the marker wires may be prone to elastic deformation or migration within the vessel. To mitigate this, the marker wire crossing points in the braid may be fused together by carefully heating the braid beyond the melting point of the absorbable polymer. This can allow the secondary marker wire to be held in place for an extended period while local tissue further integrates around secondary marker wire, which then itself begins to degrade. This fusion can also provide for a device with enhanced initial radial stiffness and strength.

The primary absorbable wire will degrade in a shorter time than the secondary absorbable marker wire. For example, the primary wire may be 50% degraded in as many as 52 weeks, 40 weeks, 30 weeks, or 20 weeks, or as little as 5 weeks, 4 weeks, 3 weeks, 2 weeks, 1 week, or less than 1 week, or within any range between any of the two foregoing values used as endpoints. The secondary marker wire will degrade in a longer time than the primary wire. For example, the secondary wire may be 50% degraded in as many as 150 weeks, 100 weeks, or 50 weeks, or as little as 40 weeks, 30 weeks, 20 weeks, 10 weeks, or 5 weeks, or within any range between any of the two foregoing values used as endpoints.

In some cases, it may be desirable to have the secondary marker wire receptive to thermal stress relieving at temperatures suitable for the primary wire. Depending on the material combinations, this may be challenging. For example, thermal stress relieving of magnesium primary wires may be ideally done as high as 450° C., 350° C., or 250° C., or as low as 200° C., 175° C., 150° C., 125° C. or 100° C., or within any temperature range between any two of the foregoing values used as endpoints. Thermal stress relieving of molybdenum, however, may be ideally done as high as 1000° C., 900° C., or 800° C., or as low as 600° C., 500° C., or 400° C., or within any temperature range between any two of the two foregoing values used as endpoints. To address this issue, it may be advantageous to prepare the marker wire from a DFT® composite wire with a magnesium alloy shell and a molybdenum core, so that the stress relief treatment for primary wires is also suitable for the secondary marker wires. A similar concept may be used for other material combinations.

### 2. Exemplary Materials and Constructs

Exemplary fully absorbable radiopaque marker wires may include molybdenum and tungsten, and alloys thereof. Others which may be suitable include zinc and iron, and alloys thereof. Still another exemplary material for the marker wire is a DFT composite with a shell of an iron-manganese alloy and a core of molybdenum.

Exemplary polymer coatings to prevent galvanic corrosion between the radiopaque marker wires and the magnesium wires include surface-eroding polymers such as polycaprolactone (PCL) and polyglycerol sebacate (PGS). Others which may be suitable include bulk eroding polymers such as PLA, PLLA, PLGA, PEG, PDS, PDO, or any other known absorbable polymer.

Exemplary constructs include tubular wire braids with 24, 32, or 48 total wires, though braids with as few as 16 and as many as 96 wires may be suitable. Of the total number of wires, as few as 1 and as many as 50% may consist of the radiopaque marker wires. A particular exemplary embodiment includes a 24 wire braid consisting of 22 Mg wires and 2 radiopaque marker wires.

### 3. Applications

Fully absorbable polymer coated radiopaque marker wires may be used in a wide variety of smaller devices deployed via a transcatheter method. This could include neurovascular stents, flow diverters, and occluders, coronary stents, peripheral stents & stent grafts, valve frames, septal occluders, and venous filters. In the gastrointestinal system, devices for the upper and lower digestive tract could benefit, as could other endoluminal devices such as biliary or ureteral stents.

### EXAMPLES

### Example 1

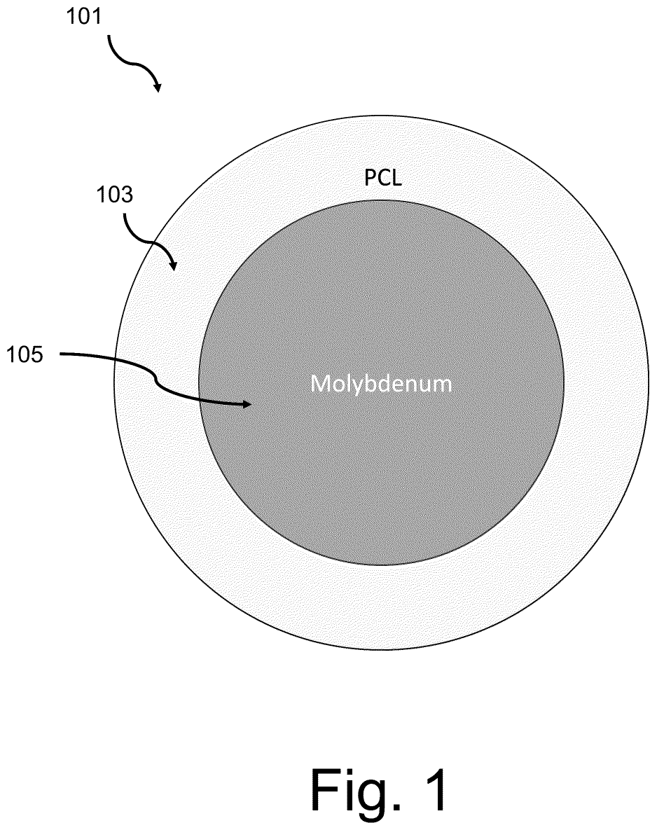

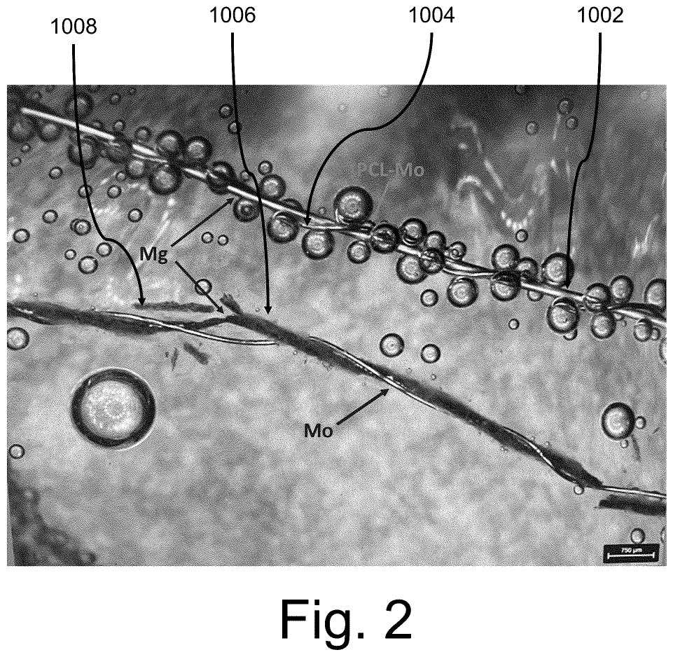

Referring to FIG. 1, an absorbable radiopaque marker wire 101, shown in cross section, was prepared as follows: 0.5 mm Mo was purchased and drawn to 0.080 mm. This wire 105 was then coated with a jacket of PCL 103 to an overall diameter of 0.120 mm. Referring to FIG. 2, to prove mitigation of galvanic effects, this PCL-Mo wire 1004 was paired with an uncoated 0.3 mm Mg alloy WE22 (Mg-2Y-1.5Nd-0.5Zn, by weight) wire 1002 by twisting and immersed in a saline solution. For comparison, the same 0.3 mm Mg alloy wire 1006 was twisted with an uncoated Mo wire 1008. After 10 minutes in the saline solution, the Mg wire paired with the PCL-Mo was corroding at a normal rate for Mg, but the Mg wire paired with the uncoated Mo wire had almost fully degraded due to the ~1.4V galvanic potential given by the Mo.

### Example 2

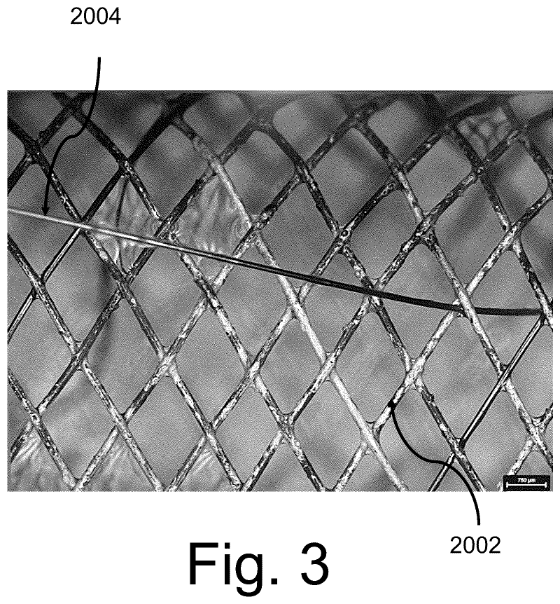

Referring to FIG. 3, a 0.080/0.120 mm Mo/PCL wire 2004 was threaded into a Mg alloy ZXM100 (Mg-1Zn-0.3Ca-0.15Mn, by weight) wire braid 2002. This assembly was placed into a saline solution and allowed to corrode for 24 hours to watch for any galvanic acceleration. None was seen.

### Example 3

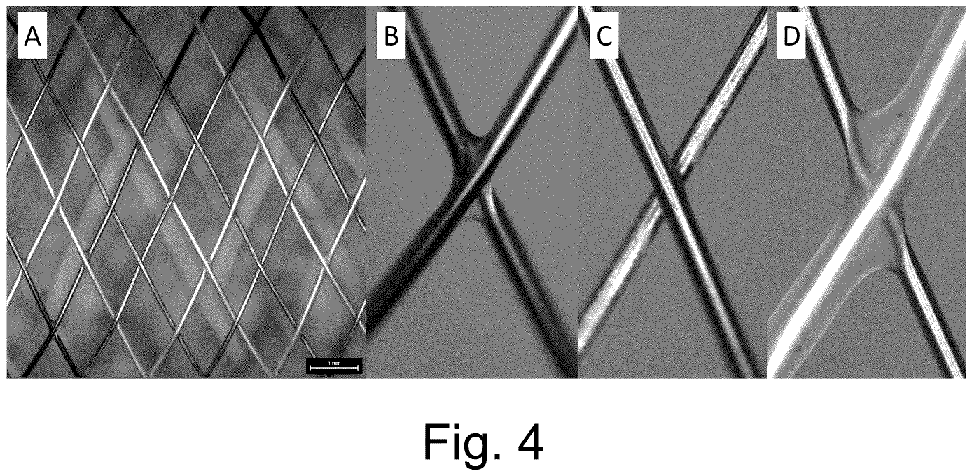

A 24-wire braid, representative of a vascular stent, was braided onto a 9.5 mm mandrel using ten 0.100 mm Mg alloy (WE22) wires, ten 0.100 mm WE22 wires coated with PCL to 0.140 mm, and four 0.080 mm Mo wires coated with PCL to 0.120 mm. The braid pattern was one over one under one, and the braid angle was set to 60°. This braid was then removed from the mandrel and suspended in a furnace set to 75° C. for 30 minutes. This allowed the PCL to melt at the crossing points and fuse the crossing points together. In FIG. 4, the overall braid structure (A), PCL-Mo/PCL-Mo crossing point (B), PCL-Mo/Mg crossing point (C), and PCL-Mo/PCL-Mg crossing point (D) can be seen.

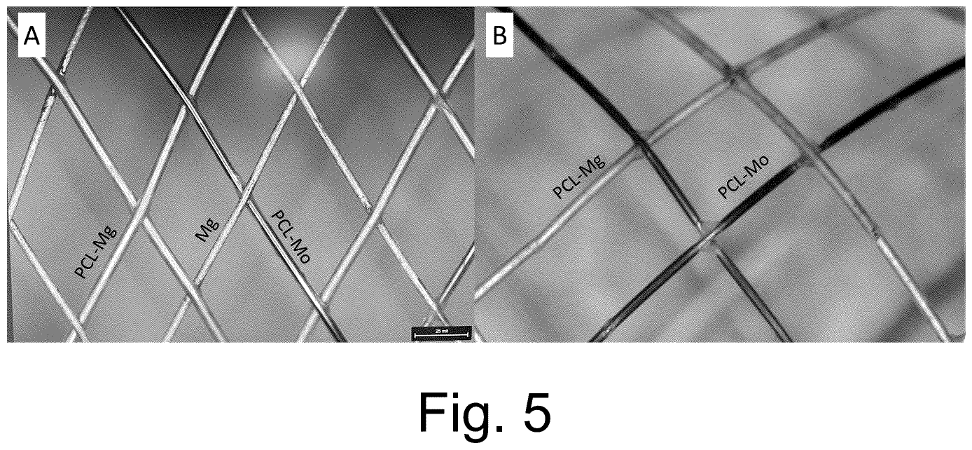

This fused braid was then immersed in a saline solution to confirm the PCL provided protection from galvanic corrosion. After 24 hours in the saline solution (FIG. 5A), the uncoated Mg wires were typically corroding. After 14 days (FIG. 5B), the uncoated Mg wires were entirely corroded and the PCL-Mg and PCL-Mo wires were still intact.

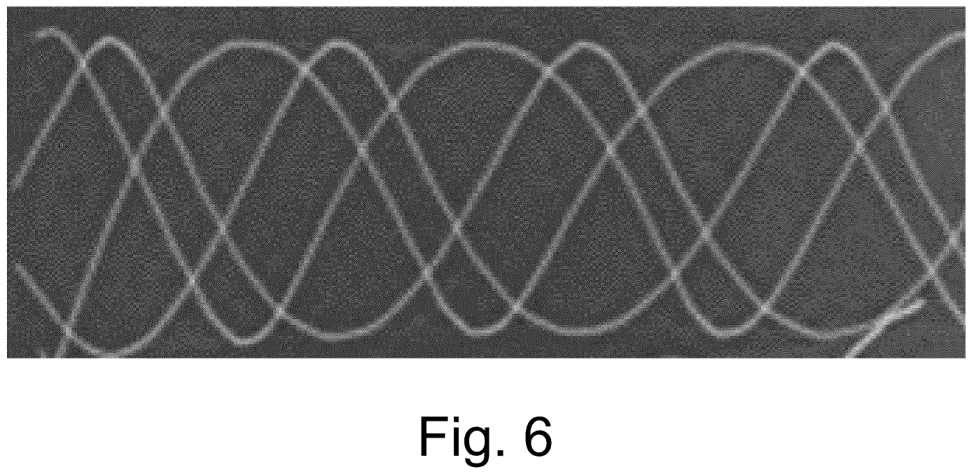

Radiopacity of the braid was confirmed via x-ray imaging (FIG. 6). The braid was placed on a 12.7 mm Al plate and imaged on a clinical x-ray scanner at 60 kV and 2.5 mAs. The PCL-Mo wires are readily visible while the Mg wires are essentially invisible, confirming the radiopacity enhancement offered by the absorbable Mo.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

### ASPECTS

Aspect 1 is a medical device, comprising a plurality of primary structural wires formed of a first absorbable metal or metal alloy; and at least one secondary marker wire formed of a radiopaque, second absorbable metal or metal alloy, each secondary marker wire coated with an absorbable polymeric material; and whereby the entire medical device is absorbable.

Aspect 2 is the medical device of Aspect 1, wherein the primary structural wire is comprised of a magnesium alloy.

Aspect 3 is the medical device of Aspect 2, wherein the coating on the secondary marker wire is comprised of one or more of PCL, PLGA, PLA, PLLA, PDO, or PGS.

Aspect 4 is the medical device of Aspect 1, wherein the secondary marker wire is comprised of commercially pure molybdenum.

Aspect 5 is the medical device of Aspect 4, wherein the coating on the secondary marker wire is comprised of one or more of PCL, PLGA, PLA, PLLA, PDO, or PGS.

Aspect 6 is the medical device of Aspect 1, wherein the secondary marker wire is comprised of zinc or a zinc alloy.

Aspect 7 is the medical device of Aspect 6, wherein the coating on the secondary marker wire is comprised of PCL, PLGA, PLA, PLLA, PDO, or PGS.

Aspect 8 is a wire for fully or partially absorbable medical devices used to enhance the device radiopacity, wherein the wire is fully absorbable and is coated with a polymer.

Aspect 9 is a medical device formed primarily of absorbable marker wires to provide whole device visibility.

Aspect 10 is a method for producing a fully absorbable and partially radiopaque medical device, comprising producing a tubular braid with a combination of primary absorbable wires and secondary polymer-coated absorbable marker wires; removing the braid from the mandrel; and heat treating the braid at a temperature slightly above the melting temperature of the absorbable polymer to fuse crossing points between the wires.

Aspect 11 is the method of Aspect 10, wherein the primary absorbable wires are a magnesium alloy, the secondary absorbable marker wires are molybdenum, and the polymer is PCL.

Aspect 12 is the method of Aspect 10, wherein the primary absorbable wires are a magnesium alloy, the secondary absorbable marker wires are molybdenum, and the polymer is PLA.

Aspect 13 is the method of Aspect 10, wherein the primary absorbable wires are a magnesium alloy, the secondary absorbable marker wires are a zinc alloy, and the polymer is PCL.

Aspect 14 is the method of Aspect 10, wherein the primary absorbable wires are a magnesium alloy, the secondary absorbable marker wires are a zinc alloy, and the polymer is PLA.

What is claimed is:

1. A medical device, comprising:
a plurality of primary structural wires formed of a first absorbable metal or metal alloy; and
at least one secondary marker wire formed of a radiopaque, second absorbable metal or metal alloy, each secondary marker wire coated with an absorbable polymeric material; and
whereby the entire medical device is absorbable.

2. The medical device of claim 1, wherein the primary structural wire is comprised of a magnesium alloy.

3. The medical device of claim 2, wherein the coating on the secondary marker wire is comprised of one or more of PCL, PLGA, PLA, PLLA, PDO, or PGS.

4. The medical device of claim 1, wherein the secondary marker wire is comprised of commercially pure molybdenum.

5. The medical device of claim 4, wherein the coating on the secondary marker wire is comprised of one or more of PCL, PLGA, PLA, PLLA, PDO, or PGS.

6. The medical device of claim 1, wherein the secondary marker wire is comprised of zinc or a zinc alloy.

7. The medical device of claim 6, wherein the coating on the secondary marker wire is comprised of PCL, PLGA, PLA, PLLA, PDO, or PGS.

8. A wire for fully or partially absorbable medical devices used to enhance the device radiopacity, wherein the wire is fully absorbable and is coated with a polymer.

9. A method for producing a fully absorbable and partially radiopaque medical device, comprising:
producing a tubular braid with a combination of primary absorbable wires and secondary polymer-coated absorbable marker wires;
removing the braid from the mandrel; and
heat treating the braid at a temperature slightly above the melting temperature of the absorbable polymer to fuse crossing points between the wires.

10. The method of claim 9, wherein the primary absorbable wires are a magnesium alloy, the secondary absorbable marker wires are molybdenum, and the polymer is PCL.

11. The method of claim 9, wherein the primary absorbable wires are a magnesium alloy, the secondary absorbable marker wires are molybdenum, and the polymer is PLA.

12. The method of claim 9, wherein the primary absorbable wires are a magnesium alloy, the secondary absorbable marker wires are a zinc alloy, and the polymer is PCL.

13. The method of claim 9, wherein the primary absorbable wires are a magnesium alloy, the secondary absorbable marker wires are a zinc alloy, and the polymer is PLA.
